(19) United States
(12) Patent Application Publication
(10) Pub. No.: US 2024/0226217 A9
(43) Pub. Date: Jul. 11, 2024

(54) METHOD FOR EXTRACTING AND PURIFYING TOTAL FLAVONOIDS FROM CARTHAMUS TINCTORIUS L. LEAF (TFFCL), AND USE THEREOF

(71) Applicant: Shaanxi University of Chinese Medicine, Shaanxi (CN)

(72) Inventors: Zhishu TANG, Shaanxi (CN); Lin CHEN, Shaanxi (CN); Zhongxing SONG, Shaanxi (CN)

(73) Assignee: Shaanxi University of Chinese Medicine, Shaanxi (CN)

(21) Appl. No.: 18/380,875

(22) Filed: Oct. 17, 2023

(30) Foreign Application Priority Data
Oct. 20, 2022 (CN) 202211289551.5

(51) Int. Cl. A61K 36/286; A61P 1/16

(52) U.S. Cl. CPC A61K 36/286; A61P 1/16

(57) ABSTRACT

The present disclosure provides a method for extracting and purifying total flavonoids from Carthamus tinctorius L. leaf, and use thereof, and belongs to the technical field of extraction and use of active ingredients of traditional Chinese medicine. The method includes: heating and refluxing Carthamus tinctorius L. leaf with ethanol to obtain a mixture, where the ethanol and Carthamus tinctorius L. leaf are at a volume ratio of (20-30):1; concentrating the mixture to form a concentrated filtrate, and adsorbing the concentrated filtrate with a macroporous resin; eluting a resulting adsorbed macroporous resin with ethanol, and collecting an obtained eluate; and concentrating the eluate until there is no residual ethanol, and drying in sequence to obtain a TFFCL powder. In the present disclosure, Carthamus tinctorius L. leaf is analyzed, the TFFCL is extracted, and applications of extracted total flavonoids (TFs) are studied, thereby better utilizing the resources of Carthamus tinctorius L.

### CROSS REFERENCE TO RELATED APPLICATION

This patent application claims the benefit and priority of Chinese Patent Application No. 202211289551.5 filed with the China National Intellectual Property Administration on Oct. 20, 2022, the disclosure of which is incorporated by reference herein in its entirety as part of the present application.

### TECHNICAL FIELD

The present disclosure belongs to the technical field of extraction and use of active ingredients of traditional Chinese medicine, and specifically relates to a method for extracting and purifying total flavonoids from Carthamus tinctorius L. leaf (TFFCL), and use thereof.

### BACKGROUND

Flos Carthami, as the dried tubular flower of an Asteraceae plant Carthamus tinctorius L., is a precious traditional medicinal material in China and one of the most widely used medicinal materials in Mongolian clinical medicine. Flos Carthami is cool in nature and slightly bitter in taste, acts towards the heart and liver meridian, and can be used for activating blood circulation and promoting menstruation, as well as removing blood stasis and relieving pain. For a long time, the use and research on Carthamus tinctorius L. have only focused on its medicinal flowers. However, Carthamus tinctorius L. plants have a poor flower yield, which greatly limits research, development, and utilization values of Carthamus tinctorius L. The medicinal flowers of Carthamus tinctorius L. are collected to analyze inherent chemical components, including alkaloids and terpenes in the medicinal flowers of Carthamus tinctorius L.

After the medicinal flowers of Carthamus tinctorius L. are harvested, a large number of Carthamus tinctorius L. leaves are discarded, resulting in a waste of resources. The leaves of Carthamus tinctorius L. also show a great application value. In addition to containing alkaloids and terpenes like the medicinal flowers of Carthamus tinctorius L., the leaves also contains flavonoids. Moreover, plant-derived flavonoids are naturally low in toxicity. However, in the prior art, the value of Carthamus tinctorius L. leaves is seldom studied, causing natural resources to be wasted.

### SUMMARY

Aiming at the deficiency that in the prior art, the value of Carthamus tinctorius L. leaves is seldom studied, causing natural resources to be wasted, the present disclosure provides a method for extracting and purifying total flavonoids from Carthamus tinctorius L. leaf (TFFCL), and use thereof.

The present disclosure, provides the extraction and purification method of TFFCL is proposed, a value of the extracted and purified TFFCL is analyzed and studied, and the TFFCL is utilized, such that the resources of Carthamus tinctorius L. may be better used. The specific technical solutions adopted by the present disclosure are as follows:

The present disclosure provides a method for extracting and purifying total flavonoids from TFFCL, including the following steps:

heating and refluxing Carthamus tinctorius L. leaf with ethanol to obtain a mixture, where the ethanol and Carthamus tinctorius L. leaf are at a volume ratio of (20-30):1; concentrating the mixture to form a concentrated filtrate, and adsorbing the concentrated filtrate with a macroporous resin; eluting a resulting adsorbed macroporous resin with ethanol, and collecting an obtained eluate; and concentrating the eluate with a rotary evaporator until there is no residual ethanol, and freezing at −80° C. overnight and drying in a vacuum freeze dryer in sequence to obtain a TFFCL powder.

In one embodiment, the heating and refluxing specifically includes: heating and refluxing Carthamus tinctorius L. leaf with the ethanol at a volume concentration of 70% to 90% 1 to 3 times for 30 min to 90 min each time.

In one embodiment, the method further includes pretreating the macroporous resin: immersing the macroporous resin with HCl at a mass concentration of 0.3% to 0.5%, and washing with water until neutral; immersing the macroporous resin with NaOH at a mass concentration of 0.3% to 0.5%, and washing with water until neutral; immersing the macroporous resin with absolute ethanol, and washing with water until the macroporous resin has no residual ethanol; and immersing the macroporous resin with 90% to 95% ethanol and then conducting wet packing.

In one embodiment, the macroporous resin is selected from the group consisting of HPD-600 macroporous resin, HPD-100 macroporous resin, D-101 macroporous resin, HP-20 macroporous resin, and AB-8 macroporous resin.

In one embodiment, the macroporous resin has a mass of 80 g to 120 g; and Carthamus tinctorius L. leaf extract in the concentrated filtrate has a volume of 100 mL to 150 mL.

In one embodiment, the method further includes adjusting the eluate to a pH value of 7.

The present disclosure further provides a TFFCL prepared by the extraction and purification method of TFFCL.

The present disclosure further provides use of the TFFCL prepared by the extraction and purification method of TFFCL in preparation of a liver-protecting drug or a liver-protecting health care product.

The present disclosure further provides use of the TFFCL prepared by the extraction and purification method of TFFCL in preparation of a drug for treating an acute liver injury.

The present disclosure further provides use of the TFFCL prepared by the extraction and purification method of TFFCL in preparation of a drug for treating a chronic liver injury.

Compared with the prior art, the present disclosure has the following beneficial effects:

1. In the present disclosure, the extraction and purification method of TFFCL extracts the TFFCL, with a simple extraction process and a high extraction efficiency. The method realizes the extraction of TFFCL, and facilitates better application of Carthamus tinctorius L. resources.

2. In the present disclosure, the macroporous resin is pretreated, such that the macroporous resin is in a neutral environment, thereby facilitating better adsorption of Carthamus tinctorius L. leaf extract in the concentrated filtrate.

3. In the present disclosure, after analysis and research, it is found that the TFFCL shows desirable application effects in liver protection. This enables further application of the TFFCL, and may significantly increase a cell viability of human liver L02 oxidatively damaged cells induced by H₂O₂(P<0.05).

4. In the present disclosure, it is found that the extracted TFFCL has an excellent application effect on acute liver injury, thereby realizing the further application of TFFCL. The TFFCL may significantly improve the liver pathological structure, liver and spleen index, liver function and other indicators in mice with the acute liver injury (P<0.05).

5. In the present disclosure, it is found that the extracted TFFCL has an excellent application effect on chronic liver injury, thereby realizing the further application of TFFCL. The TFFCL may significantly improve the liver pathological structure, liver and spleen index, liver function indicators, oxidative factors, and inflammatory factors in rats with the chronic liver injury (P<0.05).

### BRIEF DESCRIPTION OF THE DRAWINGS

FIG. 1 shows a histogram for an influence of L02 hepatocyte injury induced by H₂O₂; Note: ***P<0.001, vs control group;

FIG. 2 shows a histogram for an influence of TFFCL on L02 cytotoxicity;

FIG. 3 shows a histogram for an influence of TFFCL on a viability of L02 hepatocytes induced by H₂O₂; Note: ^{###}p<0.001, vs control group; **P<0.01, ***P<0.001, vs model group;

FIG. 4A-B show a histogram for an influence of TFFCL on a liver and spleen index of mice with CCl₄-induced chronic liver injury, where FIG. 4A is the liver index, FIG. 4B is the spleen index;

FIGS. 5A-C show an influence of TFFCL on levels of ALT, AST, and TBA in a serum of mice with CCl₄-induced chronic liver injury, where FIG. 5A is the influence of TFFCL on ALT level in the serum of mice with CCl₄-induced chronic liver injury; FIG. 5B is the influence of TFFCL on AST level in the serum of mice with CCl₄-induced chronic liver injury; and FIG. 5C is the influence of TFFCL on TBA level in the serum of mice with CCl₄-induced chronic liver injury; Note: ^{#}P<0.05, ^{###}P<0.001, vs control group; *P<0.05, **P<0.01, ***P<0.001, vs model group;

FIG. 6 shows an influence of TFFCL on pathological diagram of a liver tissue in mice with CCl₄-induced chronic liver injury, where a is appearance and morphology of the liver tissue; b is an enlarged image of HE staining after liver tissue sectioning; and c is an enlarged image of Masson staining after liver tissue sectioning;

FIG. 7 shows a histogram for an influence of TFFCL on pathological changes of the liver tissue of mice with CCl₄-induced chronic liver injury; Note: ^{###}P<0.001, vs control group; **P<0.01, ***P<0.001, vs model group;

FIGS. 8A-D show an influence of TFFCL on levels of SOD, MDA, GSH, and CAT in a liver tissue of mice with CCl₄-induced chronic liver injury, where FIG. 8A is the influence of TFFCL on SOD level in the liver tissue of mice with CCl₄-induced chronic liver injury; FIG. 8B is the influence of TFFCL on MDA level in the liver tissue of CCl₄-induced chronic liver injury; FIG. 8C is the influence of TFFCL on GSH level in the liver tissue of mice with CCl₄-induced chronic liver injury; and FIG. 8D is the influence of TFFCL on CAT level in the liver tissue of mice with CCl₄-induced chronic liver injury; Note: ^{#}P<0.01, ^{###}P<0.001, vs control group; *P<0.05, **P<0.01, ***P<0.001, vs model group;

FIGS. 9A-C show an influence of TFFCL on expression levels of IL-1β, IL-6, and TNF-α in a serum of mice with CCl₄-induced chronic liver injury, where FIG. 9A is the influence of TFFCL on IL-1β expression level in the serum of mice with CCl₄-induced chronic liver injury; FIG. 9B is the influence of TFFCL on IL-6 expression level in the serum of mice with CCl₄-induced chronic liver injury; and FIG. 9C is the influence of TFFCL on TNF-α expression level in the serum of mice with CCl₄-induced chronic liver injury; Note: ^{#}P<0.01, ^{###}P<0.001, vs control group; *P<0.05, **P<0.01, ***P<0.001, vs model group;

FIGS. 10A-B show an influence of TFFCL on the liver and spleen index of mice with acute liver injury, where FIG. 10A is the influence of TFFCL on the acute liver injury index; and FIG. 10B is the influence of TFFCL on the acute spleen injury index; Note: ^{#}P<0.05, vs control group; *P<0.05, **P<0.01, vs model group;

FIGS. 11A-C show an influence of TFFCL on biochemical criterion in a serum of mice with acute liver injury, where FIG. 11A is the influence of TFFCL on ALT level in the serum of mice with CCl₄-induced acute liver injury; FIG. 11B is the influence of TFFCL on AST level in the serum of mice with CCl₄-induced acute liver injury; and FIG. 11C is the influence of TFFCL on TBA level in the serum of mice with CCl₄-induced acute liver injury; Note: ^{###}P<0.001, vs control group; *P<0.05, **P<0.01, ***P<0.001, vs model group; and

FIGS. 12A-E show an influence of TFFCL on pathological changes of the liver tissue in mice with acute liver injury, where FIG. 12A is the influence of a blank group on the pathological changes of liver tissue in mice with acute liver injury; FIG. 12B is the influence of a model group on the pathological changes of liver tissue in mice with acute liver injury; FIG. 12C is the influence of a silymarin group on the pathological changes of liver tissue in mice with acute liver injury; FIG. 12D is the influence of a TFFCL low-dose group on the pathological changes of liver tissue in mice with acute liver injury; and FIG. 12E is the influence of a TFFCL high-dose group on the pathological changes of liver tissue in mice with acute liver injury.

### DETAILED DESCRIPTION OF THE EMBODIMENTS

The present disclosure will be further described below with reference to accompanying drawings and examples, but the present disclosure is not limited to the following examples.

### Example 1

In this example, an extraction and purification method of TFFCL included the following steps:

Carthamus tinctorius L. leaf was heated and refluxed 2 times with ethanol at a volume concentration of 80%, where Carthamus tinctorius L. leaf was fragments of Carthamus tinctorius L. leaf formed after shade-drying, crushing, and sieving, and the ethanol and Carthamus tinctorius L. leaf were at a volume ratio of 26:1; a resulting mixture was concentrated to form a concentrated filtrate, and the concentrated filtrate was adsorbed with a macroporous resin; a resulting adsorbed macroporous resin was eluted with ethanol at a volume concentration of 95%, and obtained active ingredients adsorbed by the macroporous resin were eluted into an eluate, and the eluate was collected; and the eluate was concentrated by a rotary evaporator until there was no residual ethanol, and freezed at −80° C. overnight and dried in a vacuum freeze dryer were conducted in sequence to obtain a TFFCL powder.

### Example 2

In this example, an extraction and purification method of TFFCL included the following steps:

Carthamus tinctorius L. leaf was heated and refluxed 2 times for 60 min each time with ethanol at a volume concentration of 80%, where Carthamus tinctorius L. leaf was fragments of Carthamus tinctorius L. leaf formed after shade-drying, crushing, and sieving, and the ethanol and Carthamus tinctorius L. leaf were at a volume ratio of 26.8:1; a resulting mixture by the heating reflux was concentrated to form a concentrated filtrate, where preferably, the macroporous resin had a mass of 100 g, and Carthamus tinctorius L. leaf extract in the concentrated filtrate had a volume of 120 mL; and the concentrated filtrate was adsorbed with a macroporous resin; a resulting adsorbed macroporous resin was eluted with ethanol at a volume concentration of 95%, and obtained active ingredients adsorbed by the macroporous resin were eluted into an eluate, and the eluate was collected, and then adjusted to a pH value of 7; and the eluate was concentrated by a rotary evaporator until there was no residual ethanol, and freezed at −80° C. overnight and dried in a vacuum freeze dryer were conducted in sequence to obtain a TFFCL powder.

In this example, the macroporous resin was pretreated before use, including: the macroporous resin was immersed with HCl at a mass concentration of 0.4%, and washed with water until neutral; the macroporous resin was immersed with NaOH at a mass concentration of 0.4%, and washed with water until neutral; the macroporous resin was immersed with absolute ethanol, and washed with water until the macroporous resin had no residual ethanol; and the macroporous resin was immersed with 95% ethanol and then wet packing was conducted.

In this example, the macroporous resin was HPD-600 macroporous resin, HPD-100 macroporous resin, D-101 macroporous resin, HP-20 macroporous resin, or AB-8 macroporous resin. Preferably, the macroporous resin was AB-8 macroporous resin.

In this example, the macroporous resin was washed with distilled water, or purified water, double distilled water and the like.

In this example, the volume ratio of the ethanol to Carthamus tinctorius L. leaf could also be: 20:1, 21:1, 22:1, 23:1, 24:1, 25:1, 26:1, 27:1, 28:1, 29:1, or 30:1.

### Example 3

In this example, an extraction and purification method of TFFCL included the following steps:
Carthamus tinctorius L. leaf was heated and refluxed 1 time for 90 min with ethanol at a volume concentration of 70%, where Carthamus tinctorius L. leaf was fragments of Carthamus tinctorius L. leaf formed after shade-drying, crushing, and sieving, and the ethanol and Carthamus tinctorius L. leaf were at a volume ratio of 20:1; a resulting mixture by the heating reflux was concentrated to form a concentrated filtrate, where preferably, the macroporous resin had a mass of 80 g, and Carthamus tinctorius L. leaf extract in the concentrated filtrate had a volume of 100 mL; and the concentrated filtrate was adsorbed with a macroporous resin; a resulting adsorbed macroporous resin was eluted with ethanol at a volume concentration of 90%, and obtained active ingredients adsorbed by the macroporous resin were eluted into an eluate, and the eluate was collected, and then adjusted to a pH value of 7; and the eluate was subjected to concentration by a rotary evaporator until there was no residual ethanol, and freezed at −80° C. overnight and dried in a vacuum freeze dryer were conducted in sequence to obtain a TFFCL powder.

In this example, the macroporous resin was pretreated before use, including: the macroporous resin was immersed with HCl at a mass concentration of 0.3%, and washed with water until neutral; the macroporous resin was immersed with NaOH at a mass concentration of 0.3%, and washed with water until neutral; the macroporous resin was immersed with absolute ethanol, and washed with water until the macroporous resin had no residual ethanol; and the macroporous resin was immersed with 95% ethanol and then wet packing was conducted.

In this example, the macroporous resin was HPD-600 macroporous resin, HPD-100 macroporous resin, D-101 macroporous resin, HP-20 macroporous resin, or AB-8 macroporous resin. Preferably, the macroporous resin was AB-8 macroporous resin.

In this example, the macroporous resin was washed with distilled water, or purified water, double distilled water and the like.

### Example 4

In this example, an extraction and purification method of TFFCL included the following steps:
Carthamus tinctorius L. leaf was heated refluxed 3 times for 30 min with ethanol at a volume concentration of 90%, where Carthamus tinctorius L. leaf was fragments of Carthamus tinctorius L. leaf formed after shade-drying, crushing, and sieving, and the ethanol and Carthamus tinctorius L. leaf were at a volume ratio of 30:1; a resulting mixture by the heating reflux was concentrated to form a concentrated filtrate, where preferably, the macroporous resin had a mass of 100 g, and Carthamus tinctorius L. leaf extract in the concentrated filtrate had a volume of 150 mL; and the concentrated filtrate was adsorbed with a macroporous resin; a resulting adsorbed macroporous resin was eluted with ethanol at a volume concentration of 95%, and obtained active ingredients adsorbed by the macroporous resin were eluted into an eluate, and the eluate was collected, and then adjusted to a pH value of 7; and the eluate was concentrated by a rotary evaporator until there was no residual ethanol, and freezed at −80° C. overnight and dried in a vacuum freeze dryer were conducted in sequence to obtain a TFFCL powder.

In this example, the macroporous resin was pretreated before use, including: the macroporous resin was immersed with HCl at a mass concentration of 0.5%, and washed with water until neutral; the macroporous resin was immersed with NaOH at a mass concentration of 0.5%, and washed with water until neutral; the macroporous resin was immersed with absolute ethanol, and washed with water until the macroporous resin had no residual ethanol; and the macroporous resin was immersed with 90% ethanol and then wet packing was conducted.

In this example, the macroporous resin was HPD-600 macroporous resin, HPD-100 macroporous resin, D-101 macroporous resin, HP-20 macroporous resin, or AB-8 macroporous resin. Preferably, the macroporous resin was AB-8 macroporous resin.

In this example, the macroporous resin was washed with distilled water, or purified water, double distilled water and the like.

In this application, the TFFCL referred to the total flavonoids (TFs) extracted from Carthamus tinctorius L. leaf.

The TFFCL was obtained by the extraction and purification method of TFFCL in any one of Examples 1 to 4.

In order to obtain the best extraction effect of this application, single factor experiments and response surface methods were used to optimize the extraction and purification method of TFFCL in this application, including:

Single Factor Experiments:
1. The pretreated macroporous resin was separately added to 40 mL of the concentrated filtrates in Example 2 with pH values of 2, 4, 7, 10, and 12, adsorbed by shaking for 4 h, allowed to stand for 12 h, and a total flavonoid content was detected, an adsorption rate was calculated, and an optimal pH value was selected.
2. Carthamus tinctorius L. leaf extract was taken for sampling, an effluent was collected in sections, 40 mL for each section, and 10 parts of the effluent were collected to determine the total flavonoid content. When a total flavonoid concentration in the effluent reached 10% of the concentration in the loading solution, the concentration was called a leak point. When the total flavonoid concentration in the effluent reached 100% of the concentration in the loading solution, the concentration was called a saturation point. The effluent was collected and a total flavonoid content in the effluent was determined.
3. 10 g of the macroporous resin in a saturated state after adsorbing total flavonoids was washed by deionized water, and added with 40 mL of ethanol at a volume concentration of 20%, 40%, 60%, 80%, and 95% separately to allow elution by shaking for 2 h. Each of the obtained eluates was allowed to stand for 8 h, a total flavonoid content was detected, a desorption rate was calculated, and an optimum ethanol concentration was obtained from the desorption rate.
4. 10 g of a dried powder of Carthamus tinctorius L. leaf was subjected to extraction by heating reflux, to investigate an influence of a number of extractions (1, 2, 3, 4, and 5 times), an extraction time (30, 60, 90, 120, and 150 min), a solid-to-liquid ratio (1:15, 1:20, 1:25, 1:30, and 1:35 g/mL), and an ethanol concentration (50%, 60%, 70%, 80%, and 90%) on an extraction rate of total flavonoids. The tested solution was subjected to the determination of total flavonoid content, and the extraction rate was calculated according to a standard curve.

Through the single factor experiments and comprehensive consideration of time cost and economic cost, it was concluded that the number of extraction times was 2, the solid-to-liquid ratio was 1:25, the extraction solvent was 80% ethanol, and the single extraction time was 60 min. The concentrated filtrate had an optimal pH value of 7, the optimal loading volume of the concentrated filtrate was 120 mL, the optimal volume concentration of ethanol used in the macroporous resin elution was 95%, and the purity of TFFCL obtained after enrichment and purification by AB-8 macroporous resin reached 61.42%.

Response Surface Method:

According to the single factor experiment results, under fixing the number of extractions to 2 times, 3 factors: solid-to-liquid ratio (A), ethanol concentration (B), and extraction time (C) were selected as variables, and the TFFCL extraction rate (X) was used as a response value, such that a three-factor and three-level response surface test was designed using a Box-Behnken method. Quadratic response surface regression analysis was conducted on the test data using Design-Expert8.0.6 software, to obtain the following multiple quadratic regression equation: X (%)=4.89+0.1449A+0.061B+0.0588C+0.0774AB+0.0831AC+0.0399BC−0.4463A²−0.5328B²−0.2238C². The regression model was subjected to analysis of variance: the model had a P value of <0.0001 in the significance test, indicating that the model was statistically significant; lack of fit term P was >0.05 and CV % was 1.57%, indicating that the regression model showed desirable fitting and could more accurately reflect the influence of the 3 factors on the extraction rate of TFFCL. In addition, there was a certain interaction between ethanol concentration and extraction time, and between solid-to-liquid ratio and extraction time. However, the contour lines of solid-to-liquid ratio and ethanol concentration were elliptical, and the 3D response surface had almost no curvature, indicating that there was almost no large interaction between the two factors. It was seen that the influence of various factors on the extraction rate of TFFCL was: solid-to-liquid ratio>ethanol concentration>extraction time.

The optimal extraction conditions obtained according to the response surface method included: solid-to-liquid ratio of 1:26.852, ethanol concentration of 80.776%, and extraction time of 65.175 min. Under these conditions, the TFFCL had a theoretical extraction rate of up to 4.907%. Considering the practical operability, the optimal extraction conditions were optimized as follows: the solid-to-liquid ratio of 1:27, the ethanol concentration of 80%, and the extraction time of 65 min. At this time, an actual extraction rate was 4.921%, which was 0.014% different from a theoretical value, indicating that this model had desirable and stable prediction.

The present disclosure further provided use of the TFFCL prepared by the extraction and purification method of TFFCL in preparation of a liver-protecting drug or a liver-protecting health care product.

Specifically, a TFFCL prepared by the extraction and purification method of TFFCL in the present application was used in the preparation of a drug for treating liver injury.

An experimental process mainly included H₂O₂-induced L02 liver cell injury:

H₂O₂ configuration: 1 mol/L of H₂O₂ stock solution was diluted with 1640 medium to prepare a working solution at a concentration of 250 μmol/L.

10 mg/mL of a stock solution was prepared with the TFFCL prepared in Example 2.

I. Conditional Screening of L02 Liver Cell Injury Induced by H₂O₂

L02 cells in a logarithmic growth phase were digested with trypsin, and inoculated in a 96-well culture plate at 1.5×10⁴ cells/well, 100 μL per well. 12 h after inoculation, the cells were completely attached. The original medium was separately replaced with the working solutions of 50 μmol/L, 100 μmol/L, 250 μmol/L, 500 μmol/L, and 1,000 μmol/L, and blank control wells without H₂O₂ were set up, where 6 duplicate wells were set up for each concentration and the blank control. The cells were continued to be cultured for 4 h, and 120 μL of MTT solution was added to each well (base medium:MTT=100:20), incubated for 4 h, a supernatant was discarded, and 150 μL of dimethyl sulfoxide was added. The cells were shaken for 3 min, an absorbance value was determined at 490 nm on a microplate reader, the experiment was repeated 3 times, a cell viability was calculated, and an appropriate H₂O₂ concentration range was selected to allow subsequent experiments.

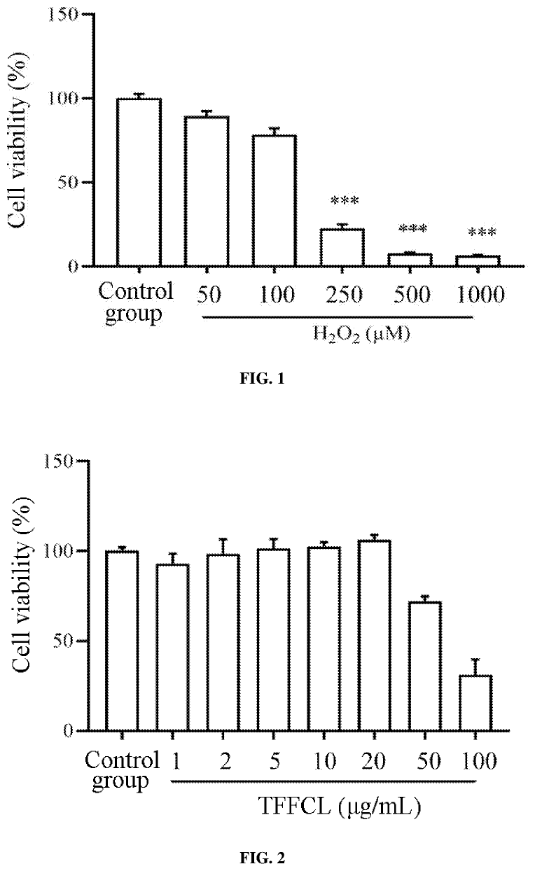

Referring to FIG. 1, compared with the control group, the cell viability of the L02 liver cells gradually decreased as the H₂O₂ concentration increased; when the concentration was 250 μmol/L, the cell viability dropped to 22.78% (P<0.001). As the concentration of hydrogen peroxide further increased, the cell viability of L02 liver cells gradually decreased, and gradually showed sparse cell growth, damaged cell membrane, and unclear cell structure. The results showed that 250 μmol/L could be selected as the condition for H₂O₂-induced L02 hepatocyte injury.

II. Influence of TFFCL on L02 Cytotoxicity

A blank control group and TFFCL treatment groups of different concentrations were set up, respectively, where the TFFCL was the product prepared in Example 2, and each group had 6 duplicate wells. L02 cells in a logarithmic growth phase were inoculated in a 96-well culture plate at 1.5×10⁴ cells/well. 12 h after inoculation, the cells were completely attached. The original medium was separately replaced with the working solutions of 1 μg·mL⁻¹, 2 μg·mL⁻¹, 5 μg·mL⁻¹, 10 μg·mL⁻¹, 20 μg·mL⁻¹, 50 μg·mL⁻¹, 100 μg·mL⁻¹, 250 μg·mL⁻¹, 500 μg·mL⁻¹, and 1,000 μg·mL⁻¹. The cells were continued to be cultured for 24 h, and 120 μL of MTT solution was added to each well (base medium:MTT=100:20), incubated for 4 h, a supernatant was discarded, and 150 μL of dimethyl sulfoxide was added. The cells were shaken for 3 min, an absorbance value was determined at 490 nm on a microplate reader, the experiment was repeated 3 times, a cell viability was calculated, and an appropriate TFFCL concentration range was selected to allow subsequent experiments.

Referring to FIG. 2, the TFFCL at a concentration of 2 μg/mL to 20 μg/mL had no cytotoxicity to L02 cells.

III. Influence of TFFCL on viability of L02 hepatocytes induced by H₂O₂

A control group, a H₂O₂ model group (at a final concentration of 250 μmol/L), and H₂O₂ with TFFCL working solution (1 μg/mL, 2 μg/mL, 5 μg/mL, 10 μg/mL, 20 μg/mL, 50 μg/mL, and 100 μg/mL), where the TFFCL was the product prepared in Example 2, and each group had 6 duplicate wells. L02 cells in a logarithmic growth phase were inoculated in a 96-well culture plate at 1.5×10⁴ cells/well. 12 h after inoculation, the cells were completely attached. The original medium was separately replaced with the working solutions containing TFFCL of 1 μg/mL, 2 μg/mL, 5 μg/mL, 10 μg/mL, 20 μg/mL, 50 μg/mL, and 100 μg/mL. The cells were pretreated for 24 h, cultured for 4 h with H₂O₂, and 120 μL of MTT solution was added to each well (base medium:MTT=100:20), incubated for 4 h, a supernatant was discarded, and 150 μL of dimethyl sulfoxide was added. The cells were shaken for 3 min, an absorbance value was determined at 490 nm on a microplate reader, the experiment was repeated 3 times, a cell viability was calculated, and an appropriate TFFCL concentration range was selected to allow subsequent experiments.

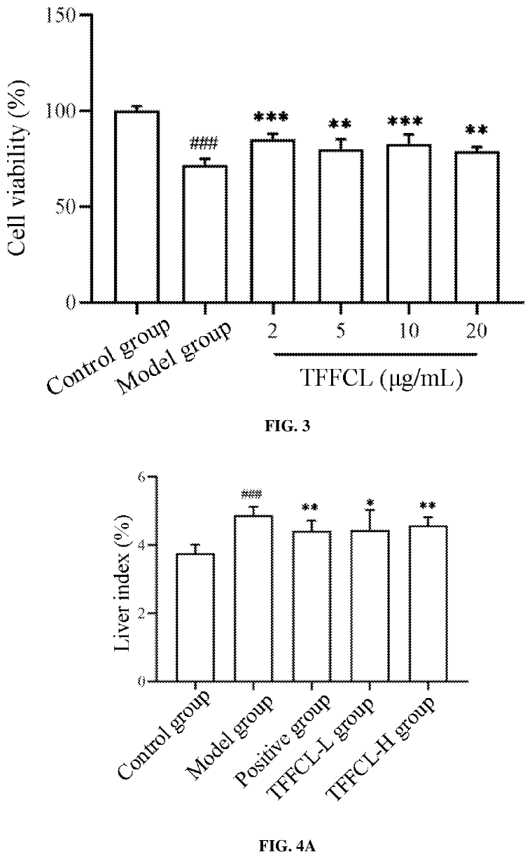

Referring to FIG. 3, compared with the control group, the 250 μmol/L H₂O₂ caused certain damages to the cell viability of L02 cells (P<0.001). Compared with the model group, the TFFCL had a certain protective effect on H₂O₂-induced oxidative damage in human liver L02 cells at 2 μg·mL⁻¹ to 20 μg·mL⁻¹; the cell viability increased from 71.9% in the model group to over 85.4%, with a significant difference (P<0.001).

The TFFCL prepared in Example 1 and Example 3 to 4 was subjected to the same H₂O₂-induced L02 liver cell injury test, and the test results were similar to the above results. That is, the TFFCL of the present application could protect the damage of liver cells, and has a certain effect in protecting the liver. The TFFCL could not only be used to prepare drugs for treating liver injury, but also be used to prepare health care products for liver injury.

Specifically, a TFFCL prepared by the extraction and purification method of TFFCL in the present application was used in the preparation of a drug for treating chronic liver injury.

An experimental process mainly included treating mice with chronic liver injury induced by carbon tetrachloride using TFFCL:

I. Influence of Long-Term Administration of TFFCL on Body Weight, Organ Indexes, and Biochemical Criterion of Mice

SPF-grade male C57BL/6 mice were randomly divided into 3 groups, 10 mice in each group. The 3 groups were labeled as a blank group, a TFFCL low-dose group, and a TFFCL high-dose group, respectively. The control group was given 0.5% carboxymethylcellulose sodium solution; the TFFCL low-dose group was given TFFCL 20 mg/kg; and the TFFCL high-dose group was given TFFCL 40 mg/kg. The mice were subjected to intragastric administration once a day continuously for 6 weeks. The body weight of the mice was monitored and recorded weekly. 16 h after the last administration at the 6th week, blood was collected from the orbit of the mice, and the liver, heart, spleen, lungs, and kidneys of the mice were weighed. The contents of biochemical criterion in mouse serum were detected by an automatic biochemical analyzer, including: alanine aminotransferase (ALT), aspartate aminotransferase (AST), alkaline phosphatase (ALP), total bile acid (TBA), albumin (ALB), total protein (TP), creatinine (CREA), urea (UREA), and uric acid (UA).

As shown in Table 1, compared with the blank group in the experimental period, the TFFCL low-dose group and the TFFCL high-dose group had no significant difference on the body weight, the organ indexes of each organ, and the serum biochemical criterion such as AST, ALT, ALP, TP, ALB, TBA, CREA, UREA and UA of the mice. This indicated that TFFCL had no significant influence on the body weight, organ indexes, and liver and kidney functions of normal mice during the administration period.

TABLE 1
Influence of long-term administration of TFFCL on body weight, organ indexes,
and serum biochemical criterion of normal mice (x̅ ± SD, n = 10)

| | | | | TFFCL |
|---|---|---|---|---|
| | | | TFFCL | high-dose |
| Index | | Control group | low-dose group | group |
| Body weight (g) | 1 week | 21.04 ± 0.61 | 21.46 ± 0.92 | 20.69 ± 0.60 |
| | 2 weeks | 21.16 ± 0.66 | 21.11 ± 0.78 | 20.51 ± 0.56 |
| | 3 weeks | 21.69 ± 0.60 | 22.13 ± 0.81 | 21.39 ± 0.88 |
| | 4 weeks | 21.43 ± 0.67 | 21.85 ± 1.19 | 21.35 ± 0.83 |
| | 5 weeks | 22.63 ± 0.68 | 22.89 ± 1.32 | 22.4 ± 1.12 |
| | 6 weeks | 23.08 ± 0.70 | 22.98 ± 1.52 | 22.52 ± 1.17 |
| Organ index (%) | Heart index (%) | 0.54 ± 0.07 | 0.51 ± 0.10 | 0.54 ± 0.09 |
| | Liver index (%) | 3.9 ± 0.12 | 4.14 ± 0.69 | 4.02 ± 0.28 |
| | Spleen index (%) | 0.32 ± 0.06 | 0.34 ± 0.05 | 0.31 ± 0.1 |
| | Lung index (%) | 0.6 ± 0.06 | 0.66 ± 0.17 | 0.63 ± 0.07 |
| | Kidney index (%) | 1.23 ± 0.09 | 1.16 ± 0.09 | 1.2 ± 0.05 |
| Serum | ALT (U/L) | 44.48 ± 3.37 | 41.90 ± 5.01 | 37.86 ± 13.55 |
| biochemical | AST (U/L) | 183.46 ± 46.11 | 186.56 ± 58.83 | 181.53 ± 44.81 |
| criterion | ALP (U/L) | 148.32 ± 22.34 | 128.79 ± 25.52 | 117.96 ± 60.10 |
| | ALB (g/L) | 32.72 ± 0.82 | 31.00 ± 1.40 | 29.36 ± 6.31 |
| | TP (g/L) | 61.12 ± 3.45 | 58.54 ± 3.44 | 52.92 ± 12.73 |
| | UA(U/L) | 105.46 ± 38.49 | 36.28 ± 18.47 | 121.10 ± 12.66 |
| | UREA(U/L) | 11.21 ± 2.19 | 11.69 ± 1.03 | 10.95 ± 1.16 |
| | TBA(U/L) | 4.74 ± 1.81 | 5.03 ± 3.58 | 5.20 ± 1.54 |
| | CREA-S(U/L) | 22.90 ± 7.93 | 30.04 ± 12.81 | 29.93 ± 14.67 |

II. Modeling of CCl₄-Induced Chronic Liver Injury and Drug Administration Test (TFFCL in this Experiment was Prepared by the Method in Example 2 of the Present Disclosure)

SPF-grade male C57BL/6 mice were randomly divided into 5 groups, 12 mice in each group. The groups were a control group, a model group, a silibinin group (positive control group), a TFFCL low-dose group, and a TFFCL high-dose group. In addition to the control group, mice in the model group, positive control group, TFFCL low-dose group, and TFFCL high-dose group were intraperitoneally injected with 20% CCl₄ (CCl₄: olive oil=1:4, 2 mL/kg); mice in the control group were intraperitoneally injected with an equal amount of olive oil solution. Each group was injected twice a week for 6 weeks. The silibinin and TFFCL were separately suspended in 0.5% CMC-Na solution. The silibinin group, TFFCL-L group (low-dose), and TFFCL-H group (high-dose) were administered silibinin 100 mg/kg, TFFCL 20 mg/kg, and TFFCL 40 mg/kg by gavage, respectively; while the control group and the model group were intragastrically administered the same amount of 0.5% CMC-Na solution. Each group was given continuous intragastric administration once a day for 6 weeks. During the animal feeding, the body weight of mice was monitored and recorded weekly; 16 h after the last administration in the 6th week, blood was collected from the orbit of the mice, the liver and spleen of the mice were weighed and photographed, and wet weights of the liver and spleen were recorded. The organ index was calculated (organ index=organ mass/body mass×100%).

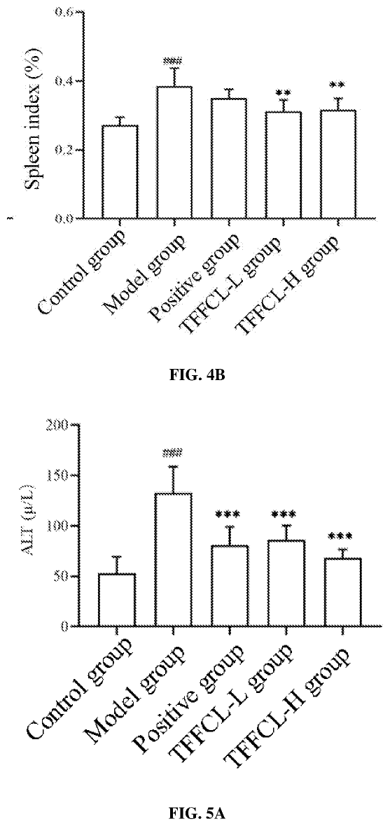

The mice in the control group had smooth coat color and desirable mental state. Compared with the control group, the mice in the model group showed listlessness, decreased appetite, and significant weight loss; the mice in the other administration groups had significantly better coat color, mental state, and food intake than those in the model group. As shown in Table 2 and FIG. 4, compared with the control group, the organ indexes of the liver and spleen of the mice in the model group increased, with extremely significant differences (P<0.001). Compared with the model group, the organ indexes of the liver and spleen in each TFFCL administration group and the positive group were significantly reduced (P<0.05).

TABLE 2
Influence of TFFCL on liver and spleen indexes
of mice with CCl₄-induced chronic liver injury

| Group | Liver index (%) | Spleen index (%) |
|---|---|---|
| Control group | 3.77 ± 0.25 | 0.27 ± 0.02 |
| Model group | 4.88 ± 0.25^{###} | 0.39 ± 0.05^{###} |
| Positive group | 4.42 ± 0.30** | 0.35 ± 0.02 |
| TFFCL low-dose group | 4.44 ± 0.59* | 0.31 ± 0.03** |
| TFFCL high-dose group | 4.59 ± 0.23** | 0.32 ± 0.03** |

Note:
^{###}P < 0.001, vs control group;
*P < 0.05,
**P < 0.01, vs model group

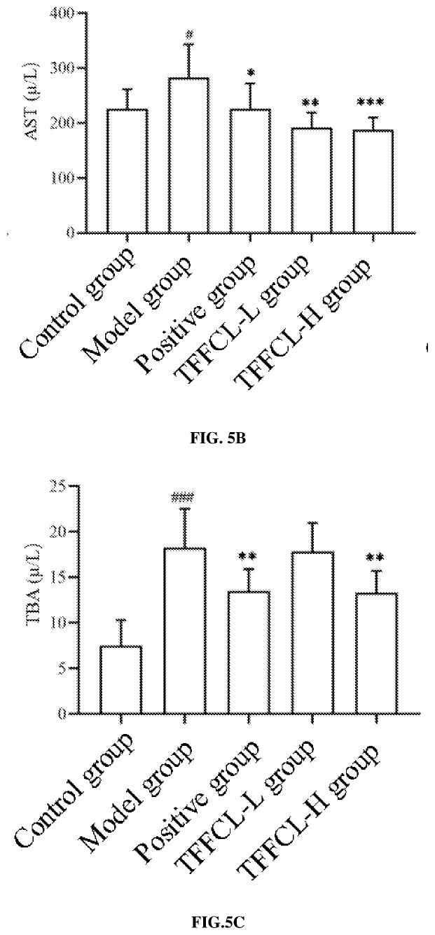

The influence of TFFCL on serum ALT, AST, and TBA levels in mice with CCl4-induced chronic liver injury were shown in Table 3 and FIG. 5. The results showed the liver biochemical index levels measured in the serum of mice in the experimental group. Compared with the control group, the ALT and TBA levels of the mice in the model group were significantly increased (P<0.001), and the AST level was significantly increased (P<0.05). It indicated that CCl₄ successfully induced the liver injury in mice. Compared with the model group, the ALT and AST levels of the TFFCL low-dose group and TFFCL high-dose group dropped to below 64.3 and 67.8% of the model group, respectively, with significant differences (P<0.05). The TBA level of the TFFCL high-dose group dropped to 72.9% of that of the model group, with a significant difference (P<0.05). The results showed that the TFFCL could better protect liver cells and improve liver functions.

TABLE 3
Influence of TFFCL on serum ALT, AST, and TBA levels of mice with CCl₄-induced
chronic liver injury (x̅ ± SD, n = 10)

| Group | ALT (U/L) | AST (U/L) | TBA (U/L) |
|---|---|---|---|
| Control group | 52.86 ± 12.37 | 226.05 ± 35.65 | 7.51 ± 2.79 |
| Model group | 132.86 ± 25.64^{###} | 283.09 ± 60.50^{#} | 18.29 ± 4.23^{###} |
| Positive group | 80.58 ± 18.26*** | 226.58 ± 45.39* | 13.54 ± 2.37** |
| TFFCL low-dose group | 86.10 ± 14.08*** | 192.05 ± 27.30** | 17.86 ± 3.11 |
| TFFCL high-dose group | 68.26.39 ± 8.51*** | 188.21.77 ± 22.20*** | 13.33 ± 2.40** |

Note:
^{#}P < 0.05,
^{###}P < 0.001, vs control group;
**P < 0.01,
***P < 0.001, vs model group

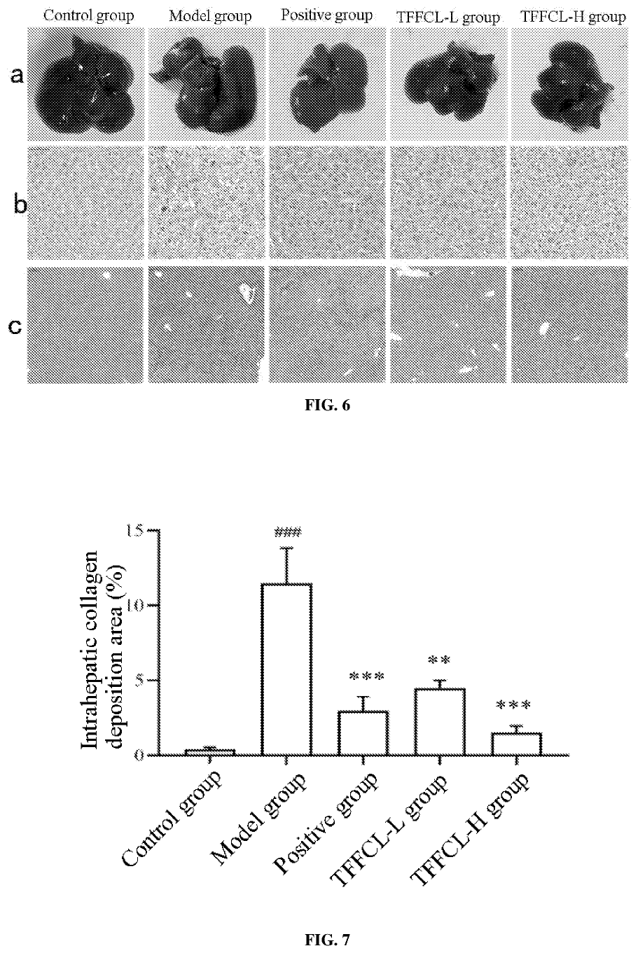

The influence of TFFCL on the pathological changes of liver tissue in mice with chronic liver injury caused by CCl₄ were shown in FIG. 6 and FIG. 7. The livers of mice in the control group had a smooth surface and ruddy color; the livers of the mice in the model group were dull in color, less glossy, with obvious coarse granular substances on the surface, and blunted edges; the livers of the mice in the positive group and the TFFCL administration groups still had fine granular substances on the surface, but the livers were improved to varying degrees compared with those in the model group. HE staining is a common pathological detection method for determine the state of liver injury such as inflammatory cell infiltration, cellular fatty degeneration, and necrosis. The results of HE staining showed that the liver tissue of the mice in the control group had a normal structure, and the liver cells were arranged neatly and uniformly in size without necrosis, and arranged radially around the central vein; the liver cell nucleus was located in the center of the cell, round, with clear boundaries, and the structure of the liver lobules was intact. The liver cells of the mice in the model group were arranged in disorder, showed fibrous connective tissue hyperplasia, and inflammatory cells and necrotic cells increased in the portal area; there was a large amount of steatosis and hepatocyte necrosis, as well as obvious inflammatory cell infiltration. The degeneration, necrosis, and inflammatory cell infiltration of liver cells in mice in the positive group, TFFCL low-dose group, and TFFCL high-dose group were significantly improved compared with those in the model group.

Masson staining is generally used to identify collagen fibers and muscle fibers, and can accurately determine the degree of fibrosis in liver tissue. Masson staining results showed that the cells in liver tissue sections of mice in the control group had a complete structure, and no obvious collagen fibers were produced; a large number of hyperplastic collagen fibers were clearly observed in the liver tissue sections of the mice in the CCl₄ group; the liver tissue sections of mice in the positive group, TFFCL low-dose group, and TFFCL high-dose group still had a certain amount of collagen fiber formation, which was lighter than those in the model group. The TFFCL low-dose group and the TFFCL high-dose group could reduce collagen fibrous tissue proliferation and fibrosis to varying degrees. According to the statistics of a positive area of collagen fibers in the livers of mice in each group, the positive area of collagen fibers in the model group increased from 1.01% of the blank group to 9.52%, and there was a significant difference between the two groups (P<0.001); compared with the model group, the positive area of collagen fibers in both the positive group and the TFFCL group decreased to less than 3.47%, and there was a significant difference between the two groups (P<0.01). The results showed that administration of TFFCL could reduce collagen fibrous tissue hyperplasia and fibrosis to varying degrees.

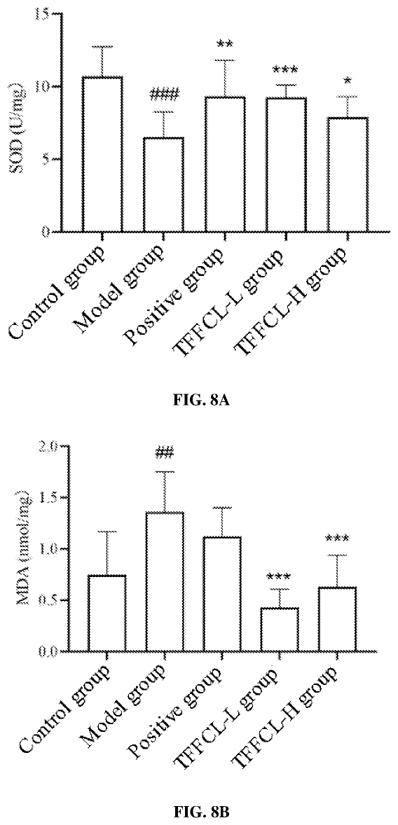

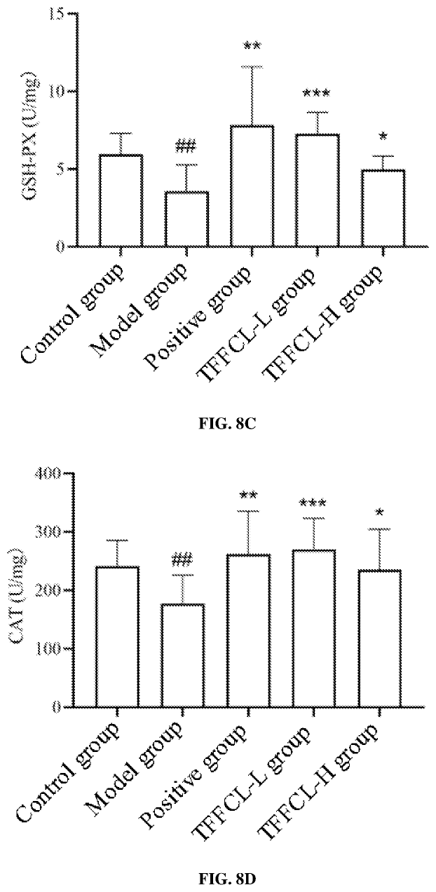

As shown in Table 4 and FIG. 8: compared with the control group, in the liver tissue of the model group, the contents of SOD, CAT, and GSH were significantly reduced (p<0.01), and the content of MDA was significantly increased (P<0.01). Compared with the model group, the TFFCL group and the positive drug group could improve the liver tissue oxidation indexes of mice with chronic liver injury to varying degrees (P<0.05). The results indicated that TFFCL could exert hepatoprotective effects through antioxidant activity.

TABLE 4
Influence of TFFCL on liver tissue SOD, MDA, GSH, and CAT
levels of mice with CCl₄-induced chronic liver injury

| Group | SOD (U/mg) | MDA (nmol/mg) | GSH-PX (U/mg) | CAT (U/mg) |
|---|---|---|---|---|
| Control group | 10.71 ± 2.05 | 0.75 ± 0.42 | 5.95 ± 1.35 | 241.83 ± 43.91 |
| Model group | 6.53 ± 1.73^{###} | 1.36 ± 0.39^{##} | 3.58 ± 1.72^{##} | 177.91 ± 48.17^{##} |
| Positive group | 9.33 ± 2.47** | 1.12 ± 0.28 | 7.84 ± 3.75** | 262.23 ± 73.45** |
| TFFCL low-dose group | 9.26 ± 0.84*** | 0.43 ± 0.18*** | 7.28 ± 1.36*** | 270.42 ± 53.03*** |
| TFFCL high-dose group | 7.90 ± 1.40* | 0.63 ± 0.31*** | 4.99 ± 0.85* | 235.61 ± 69.57* |

Note:
^{#}P < 0.05,
^{###}P < 0.001, vs control group;
*P < 0.05,
**P < 0.01,
***P < 0.001, vs model group

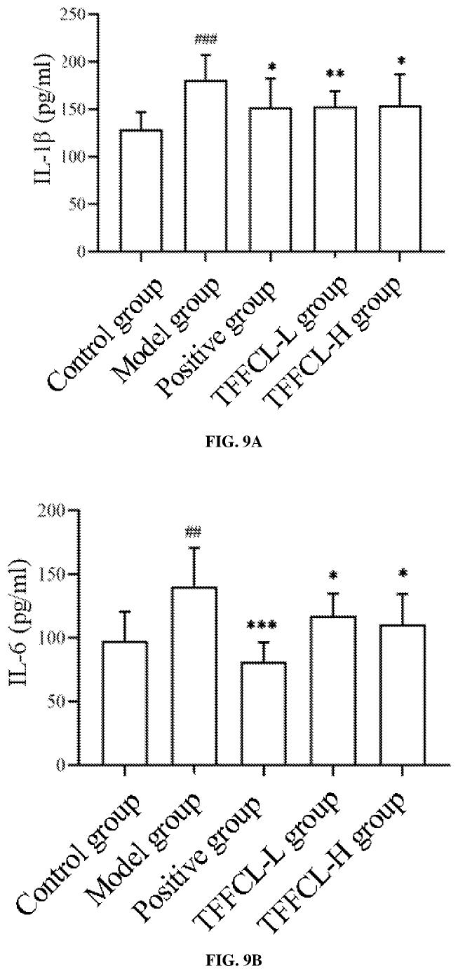

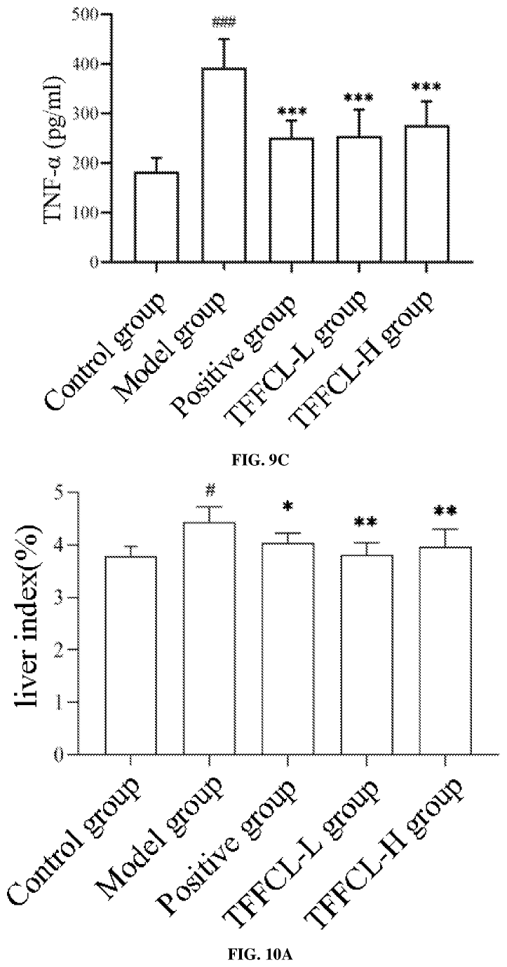

The influence of TFFCL on serum inflammatory indicators in mice with chronic liver injury caused by CCl₄ was shown in Table 5 and FIG. 9. Compared with the control group, the levels of TNF-α, IL-1β, and IL-6 in the serum of the model group were significantly increased (P<0.001). Compared with the model group, the TFFCL-L group significantly inhibited the expression of TNF-α, IL-1β, and IL-6 (P<0.01); the positive group and TFFCL-H group could reduce the levels of TNF-α, IL-1β, and IL-6 to varying degrees (P<0.05). The above results preliminarily indicated that the TFFCL low-dose group and the high-dose TFFCL group could inhibit the expression of inflammatory factors in the serum of mice with liver fibrosis in a certain concentration range by regulating immunity. In this way, the TFFCL exerted an anti-hepatic fibrosis effect.

TABLE 5
Influence of TFFCL on expression levels of IL-1β, IL-6, and TNF-
α in serum of mice with CCl₄-induced chronic liver injury

| Group | IL-1β (pg/mL) | IL-6 (pg/mL) | TNF-α (pg/mL) |
|---|---|---|---|
| Control group | 128.91 ± 18.08 | 97.91 ± 22.74 | 183.18 ± 27.51 |
| Model group | 181.21 ± 25.88^{###} | 140.39 ± 30.40^{##} | 392.72 ± 57.92^{###} |
| Positive group | 152.13 ± 30.49* | 81.40 ± 15.12*** | 252.32 ± 34.16*** |
| TFFCL low-dose group | 153.26 ± 15.80** | 117.43 ± 17.54* | 255.33 ± 53.01*** |
| TFFCL high-dose group | 154.55 ± 32.42* | 110.68 ± 23.87** | 277.46 ± 47.454*** |

Note:
^{#}P < 0.05,
^{###}P < 0.001, vs control group;
*P < 0.05,
**P < 0.01,
***P < 0.001, vs model group

The TFFCL prepared in Example 1 and Example 3 to 4 was subjected to the same chronic liver injury test induced by CCl₄, and the test results were similar to the above results. That is, the TFFCL of the present application could protect the chronic damage of liver cells, improve liver functions, and reduce the proliferation and fibrosis of collagen fibers in liver tissue. In addition, the TFFCL had an antioxidant activity and liver-protective effects, and could be used not only to prepare drugs for treating chronic liver injury, but also to prepare health care products for chronic liver injury.

Specifically, a TFFCL prepared by the extraction and purification method of TFFCL in the present application was used in the preparation of a drug for treating acute liver injury.

An experimental process mainly included treating mice with acute liver injury induced by carbon tetrachloride using TFFCL:

50 healthy male Kunming mice were randomly divided into a blank group, a model group, a silymarin group (positive control group), a TFFCL low-dose (TFFCL-L) group, and a TFFCL high-dose (TFFCL-H) group, with 10 mice in each group. The TFFCL-L group and TFFCL-H group were given 50 mg/kg and 100 mg/kg of the TFFCL extract prepared in Example 2, respectively; the silymarin group was given 100 mg/kg of silymarin; the blank group and the model group were given an equal volume of 0.5% carboxymethylcellulose sodium solution by intragastric administration. Each group was administered by intragastric administration once a day for 14 d. At the end of the 14th day, in addition to the mice in the blank group that were intraperitoneally injected with an equal amount of olive oil solution, the mice in the model group, silymarin group, TFFCL-L group, and TFFCL-H group were all intraperitoneally injected with 10 mL/kg of an olive oil solution containing 0.1% CCl₄. After the last administration of the mice, blood was collected from the eyeballs at intervals of 24 h and the mice were sacrificed. Their liver and spleen were separated and weighed, and the liver index and spleen index of the mice were calculated; the serum was collected, and the contents of ALT, AST, and TBA in mouse serum were detected by an automatic biochemical analyzer.

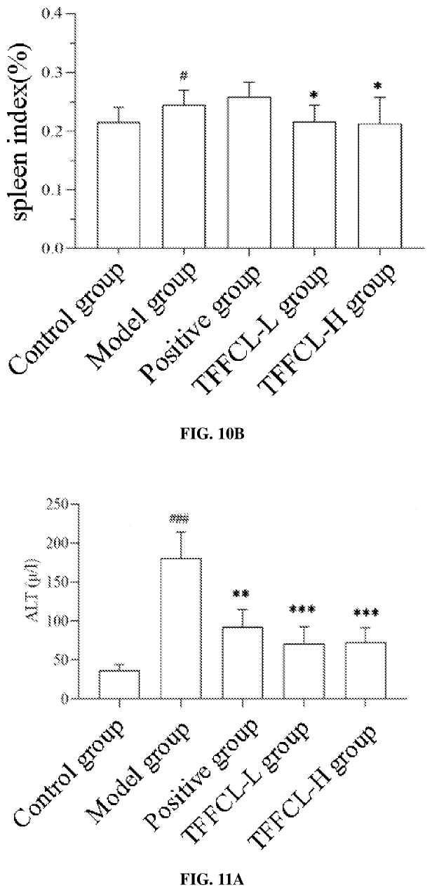

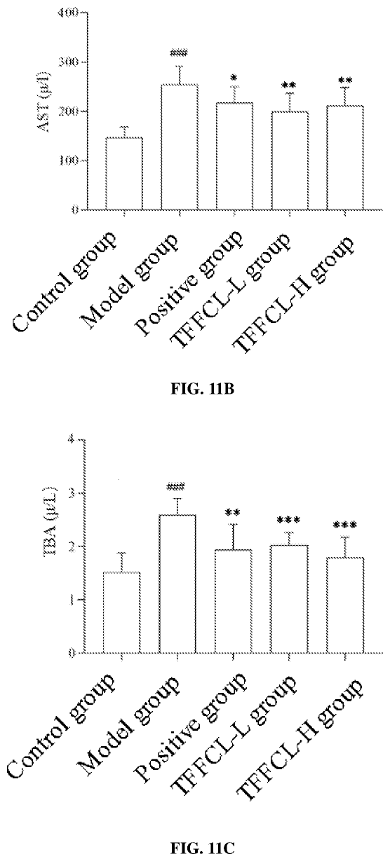

The body weight of the mice was monitored and recorded every week. After the experiment, the liver and spleen of the mice in each group were removed, washed with normal saline, dried with filter paper, photographed and weighed, and the wet weights of the liver and spleen were recorded. The organ index was calculated (organ index=organ mass/body mass×100%), to verify the influence of TFFCL on the liver and spleen index of mice with acute liver injury. Referring to FIG. 10, the mice in the blank group had smooth coat color and desirable mental state. Compared with the control group, the mice in the model group showed listlessness, decreased appetite, and significant weight loss; the remaining mice in the silymarin group, TFFCL-L group, and TFFCL-H group had significantly better coat color, mental state, and food intake than those in the model group. Compared with the control group, the organ indexes of the liver and spleen of the mice in the model group increased, with significant differences (P<0.05). Compared with the model group, the liver index of the silymarin group was significantly reduced (P<0.05), and the liver and spleen indexes of the TFFCL-L group and TFFCL-H group were reduced to varying degrees (P<0.05).

The levels of AST, ALT, and TBA in the serum of mice in each group were monitored; referring to FIG. 11, compared with the blank, the levels of ALT, AST, and TBA in mice in the model group were significantly increased (P<0.001). This indicated that the mice in the model group suffered liver function damage. Compared with the model group, the ALT, AST, and TBA levels of mice in the silymarin group, TFFCL-L group, and TFFCL-H group were significantly reduced (P<0.05), indicating that the TFFCL could better improve the liver function of mice with acute liver injury.

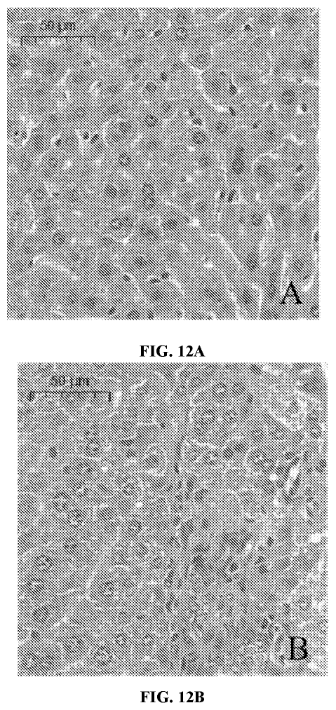

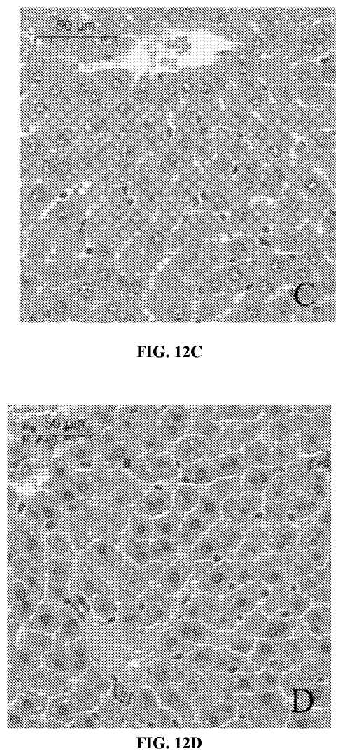

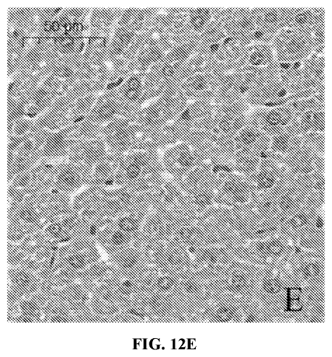

The livers of mice in each group were fixated in 4% paraformaldehyde fixative. The liver tissues were embedded in paraffin and sectioned (5 μm), dewaxed in a conventional manner, stained with hematoxylin-eosin, and observed under a microscope for pathological changes of liver tissue injury. The results of HE staining were shown in FIG. 12, the liver tissue of mice in the blank group had a normal structure, and the liver cells were arranged neatly and uniformly in size without necrosis, and arranged radially around the central vein; the liver cell nucleus was located in the center of the cell, round, with clear boundaries, and the structure of the liver lobules was intact. The liver cells of the mice in the model group were arranged in disorder, showed fibrous connective tissue hyperplasia, and inflammatory cells and necrotic cells increased in the portal area; there was a large amount of steatosis and hepatocyte necrosis, as well as obvious inflammatory cell infiltration. The degeneration, necrosis, and inflammatory cell infiltration of liver cells in mice in the silymarin group, TFFCL-L group, and TFFCL-H group were significantly improved compared with those in the model group.

The TFFCL prepared in Example 1 and Examples 3 to 4 were subjected to the same test of acute liver injury induced by CCl₄, and the test results were similar to the above results. That is to say, the TFFCL of the present application could protect the acute injury of liver cells and improve the acute liver injury of mice. The TFFCL could also improve liver cell degeneration, liver cell necrosis, and liver cell inflammation in mice. The TFFCL had a liver-protecting effect and could be used not only to prepare drugs for treating acute liver injury, but also to prepare health care products for acute liver injury.

1. A method for extracting and purifying total flavonoids from Carthamus tinctorius L. leaf (TFFCL), comprising the following steps:
heating and refluxing Carthamus tinctorius L. leaf with ethanol to obtain a mixture, wherein the ethanol and Carthamus tinctorius L. leaf are at a volume ratio of (20-30):1; concentrating the mixture to form a concentrated filtrate, and adsorbing the concentrated filtrate with a macroporous resin; eluting a resulting adsorbed macroporous resin with ethanol, and collecting an obtained eluate; and concentrating the eluate until there is no residual ethanol, and freezing and drying in sequence to obtain a TFFCL powder.

2. The method according to claim 1, wherein the heating and refluxing specifically comprises: heating and refluxing Carthamus tinctorius L. leaf with the ethanol at a volume concentration of 70% to 90% 1 to 3 times for 30 min to 90 min each time.

3. The method according to claim 1, further comprising pretreating the macroporous resin: immersing the macroporous resin with HCl at a mass concentration of 0.3% to 0.5%, and washing with water until neutral; immersing the macroporous resin with NaOH at a mass concentration of 0.3% to 0.5%, and washing with water until neutral; immersing the macroporous resin with absolute ethanol, and washing with water until the macroporous resin has no residual ethanol; and immersing the macroporous resin with 90% to 95% ethanol and then conducting wet packing.

4. The method according to claim 1, wherein the macroporous resin is selected from the group consisting of HPD-600 macroporous resin, HPD-100 macroporous resin, D-101 macroporous resin, HP-20 macroporous resin, and AB-8 macroporous resin.

5. The method according to claim 1, wherein the macroporous resin has a mass of 80 g to 120 g; and Carthamus tinctorius L. leaf extract in the concentrated filtrate has a volume of 100 mL to 150 mL.

6. The method according to claim 1, further comprising adjusting the eluate to a pH value of 7.

7. A method for preparing a liver-protecting drug or a liver-protecting health care product using TFFCL prepared by the method for extracting and purifying TFFCL according to claim 1.

8. A method for treating liver injury, comprising administering TFFCL prepared by the method for extracting and purifying TFFCL according to claim 1 to a subject with liver injury.

9. The method according to claim 8, the liver injury comprises acute liver injury and chronic liver injury.

10. (canceled)

11. The method according to claim 4, further comprising pretreating the macroporous resin: immersing the macroporous resin with HCl at a mass concentration of 0.3% to 0.5%, and washing with water until neutral; immersing the macroporous resin with NaOH at a mass concentration of 0.3% to 0.5%, and washing with water until neutral; immersing the macroporous resin with absolute ethanol, and washing with water until the macroporous resin has no residual ethanol; and immersing the macroporous resin with 90% to 95% ethanol and then conducting wet packing.

12. The method according to claim 7, wherein the heating and refluxing specifically comprises: heating and refluxing Carthamus tinctorius L. leaf with the ethanol at a volume concentration of 70% to 90% 1 to 3 times for 30 min to 90 min each time.

13. The method according to claim 7, the method for extracting and purifying TFFCL further comprising pretreating the macroporous resin: immersing the macroporous resin with HCl at a mass concentration of 0.3% to 0.5%, and washing with water until neutral; immersing the macroporous resin with NaOH at a mass concentration of 0.3% to 0.5%, and washing with water until neutral; immersing the macroporous resin with absolute ethanol, and washing with water until the macroporous resin has no residual ethanol; and immersing the macroporous resin with 90% to 95% ethanol and then conducting wet packing.

14. The method according to claim 8, wherein the heating and refluxing specifically comprises: heating and refluxing Carthamus tinctorius L. leaf with the ethanol at a volume concentration of 70% to 90% 1 to 3 times for 30 min to 90 min each time.

15. The method according to claim 8, the method for extracting and purifying TFFCL further comprising pretreating the macroporous resin: immersing the macroporous resin with HCl at a mass concentration of 0.3% to 0.5%, and washing with water until neutral; immersing the macroporous resin with NaOH at a mass concentration of 0.3% to 0.5%, and washing with water until neutral; immersing the macroporous resin with absolute ethanol, and washing with water until the macroporous resin has no residual ethanol; and immersing the macroporous resin with 90% to 95% ethanol and then conducting wet packing.
